(19) United States
(12) Patent Application Publication
(10) Pub. No.: US 2008/0312555 A1
(43) Pub. Date: Dec. 18, 2008

(54) Devices and methods for glucose measurement using rechargeable battery energy sources

(76) Inventor: Dirk Boecker, Palo Alto, CA (US)

Correspondence Address: Goodwin Procter LLP;Attn: Patent Administrator, 135 Commonwealth Drive, Menlo Park, CA, 94025-1105, US

(21) Appl. No.: 12/128,444

(22) Filed: May 28, 2008

Related U.S. Application Data
(63) Continuation-in-part of application No. 10/574,373, filed on Dec. 3, 2007.

(51) Int. Cl. A61B 5/151

(52) U.S. Cl. 600/583; 600/365

(57) ABSTRACT

An analyte measurement device has a housing and a visual display on said housing. The visual display has at lease one visual indicator position next to a corresponding marking on the housing. A processor drives the visual display and runs software that is modifiable to provide a variable user interface on the visual display. A rechargeable battery is provided.

### CROSS-REFERENCE TO RELATED APPLICATIONS

This application is a continuation-in-part of U.S. Ser. No. 10/574,373 filed Oct. 14, 2004, which application is a §3.71 filing of PCT/US04/3415, filed Oct. 14, 2003, both of which applications are fully incorporated herein by reference.

### BACKGROUND OF THE INVENTION

1. Field of the Invention

This invention relates generally to glucose measurement devices, and more particularly to glucose measurement devices that have rechargeable batteries.

2. Description of the Related Art

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet. These include cantilever springs, diaphragms, coil springs, as well as gravity plumbs used to drive the lancet The device may be held against the skin and mechanically triggered to ballistically launch the lancet.

Unfortunately, the pain associated with each lancing event using known technology discourages patients from testing. In addition to vibratory stimulation of the skin as the driver impacts the end of a launcher stop, known spring based devices have the possibility of harmonically oscillating against the patient tissue, causing multiple strikes due to recoil. This recoil and multiple strikes of the lancet against the patient is one major impediment to patient compliance with a structured glucose monitoring regime.

Another impediment to patient compliance is the lack of spontaneous blood flow generated by known lancing technology. In addition to the pain as discussed above, a patient may need more than one lancing event to obtain a blood sample since spontaneous blood generation is unreliable using known lancing technology. Thus the pain is multiplied by the number of tries it takes to successfully generate spontaneous blood flow. Different skin thickness may yield different results in terms of pain perception, blood yield and success rate of obtaining blood between different users of the lancing device. Known devices poorly account for these skin thickness variations.

A still further impediment to improved compliance with glucose monitoring are the many steps and hassle associated with each lancing event. Many diabetic patients that are insulin dependent may need to self-test for blood glucose levels five to six times daily.

The large number of steps required in traditional methods of glucose testing, ranging from lancing, to milking of blood, applying blood to the test strip, and getting the measurements from the test strip, discourages many diabetic patients from testing their blood glucose levels as often as recommended. Older patients and those with deteriorating motor skills encounter difficulty loading lancets into launcher devices, transferring blood onto a test strip, or inserting thin test strips into slots on glucose measurement meters. Additionally, the wound channel left on the patient by known systems may also be of a size that discourages those who are active with their hands or who are worried about healing of those wound channels from testing their glucose levels.

Additionally, known glucose meters have user interfaces that are specific to that particular meter. They contain certain features and those features are not changeable.

Manufacturers decide which user interface goes with which feature set. As soon as that part is decided, the device is set. They cannot mix and match with ease. This creates a large number of products, each directed at specific target customers. This increases the cost of the meters since they are produced at low volumes, each targeted for particular, niche user group.

Additionally, the market has not accepted the Palm or other PDA devices coupled to meters since very small numbers of diabetics actually use computers to monitor their illness. Devices of such nature have low market penetration. Basing a meter on another company's technology is questionable since the pace of advancement is so fast that devices are advancing quickly and sometimes are no longer supported by the time a diabetes monitoring program or application is ready. These advances obsolete devices and systems designed to piggyback off of other devices.

Accordingly, there is a need for an analyte measurement device that has a disposable battery. There is a further need for an analyte measurement device with a rechargeable battery that is periodically connecting the portable lancing aid to a charging station.

### SUMMARY OF THE INVENTION

An object of the present invention is to provide devices and method for analyte detection using a disposable battery.

Another object of the present invention is to provide devices and methods for analyte detection using a device with a rechargeable battery that is periodically connected to a charging station.

A further object of the present invention is to provide devices and methods for analyte detection using a device with a rechargeable battery that becomes partially depleted and is periodically recharged from a port of the device at a charging station.

These and other objects of the present invention are achieved in an analyte measurement device with a housing, and a visual display on said housing. The visual display has at lease one visual indicator position next to a corresponding marking on the housing. A processor drives the visual display and runs software that is modifiable to provide a variable user interface on the visual display. A rechargeable battery is provided.

In another embodiment, a method of periodically obtaining blood samples is provided. An electrically powered portable lancing aid is used to periodically puncture a body part and obtaining a blood sample from the body part. An energy source of the portable lancing aid is at least partially depleted and periodically connected to a charging station to recharge the energy source.

In another embodiment, an analyte measurement device includes a housing, a visual display on the housing and a penetrating member configured to be coupled to an actuator. A port is provided for recharging a power source positioned in the housing.

In another embodiment of the present invention, a skin lancing device includes a housing and a visual display on the housing. The visual displays has at least one visual indicator position next to a corresponding marking on the housing. A processor drives the visual display and runs software that is modifiable to provide a variable user interface on the visual display. A penetrating member is coupled to an actuator. A port is provided for recharging a battery positioned in the housing.

In another embodiment, a skin lancing device is provided that includes a battery and a port to recharge the battery. Software is downloaded to a monitor. The software contains a selected user interface. A driver is used to periodically drive a penetrating member, pierce a skin surface and create a wound tract. The port is used periodically to recharge the battery.

In another embodiment, a method is provided for periodically obtaining blood samples. An electrically powered portable skin lancing device is used to puncture a body part and obtain a blood sample from the body part. The energy source of the portable skin lancing device is partially depleted and periodically connected to a charging station to recharge a battery of the skin lancing device.

In another embodiment, a method is provided for periodically obtaining blood samples. An electrically powered skin lancing device is used periodically to puncture a body part and obtain a blood sample from the body part. The skin lancing device includes a port for recharging a battery positioned in the skin lancing device. The port is used to recharge the battery.

In another embodiment, a skin lancing device is provided that includes a plurality of penetrating members, a plurality of analyte sensors, a driver, a battery and a port for recharging the battery. A driver is used periodally to drive a penetrating member, pierce a skin surface and create a wound tract. The port is used periodically to recharge the battery.

### BRIEF DESCRIPTION OF THE DRAWINGS

FIG. 1 illustrates an embodiment of a controllable force driver in the form of a cylindrical electric penetrating member driver using a coiled solenoid-type configuration.

FIG. 2A illustrates a displacement over time profile of a penetrating member driven by a harmonic spring/mass system.

FIG. 2B illustrates the velocity over time profile of a penetrating member driver by a harmonic spring/mass system.

FIG. 2C illustrates a displacement over time profile of an embodiment of a controllable force driver.

FIG. 2D illustrates a velocity over time profile of an embodiment of a controllable force driver.

FIG. 3 is a diagrammatic view illustrating a controlled feed-back loop.

FIG. 4 is a perspective view of a tissue penetration device having features of the invention.

FIG. 5 is an elevation view in partial longitudinal section of the tissue penetration device of FIG. 4

FIGS. 6 through 9 show various views of the embodiments of the device of FIG. 6.

FIGS. 10-11 show a perspective view of one embodiment of the present invention.

FIG. 12 shows a view of one embodiment of the present invention.

FIGS. 13-17 illustrate various elements that may be shown on a display of the present invention.

FIG. 18 shows one embodiment of the present invention configured for wireless communication.

FIG. 19 shows an embodiment of the present invention using a stylet.

FIG. 20 is a flow chart showing one embodiment of a user interface selection process.

FIG. 21 shows an embodiment of the device with a keyboard.

FIG. 22 is a graph showing time usage during analyte measurement.

FIG. 23 shows a system with a computer and a device according to the present invention.

FIGS. 24A and 24B show other embodiments of the present invention.

### DETAILED DESCRIPTION

The present invention provides a solution for body fluid sampling. Specifically, some embodiments of the present invention provides a penetrating member device for consistently creating a wound with spontaneous body fluid flow from a patient. The invention may be a multiple penetrating member device with an optional high density design. It may use penetrating members of smaller size than known penetrating members. The device may be used for multiple lancing events without having to remove a disposable from the device or for the user to handle sharps. The invention may provide improved sensing capabilities. At least some of these and other objectives described herein will be met by embodiments of the present invention.

It is to be understood that both the foregoing general description and, the following detailed description are exemplary and explanatory only and are not restrictive of the invention, as claimed. It should be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings: “Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

“Analyte detecting member” refers to any use, singly or in combination, of chemical test reagents and methods, electrical test circuits and methods, physical test components and methods, optical test components and methods, and biological test reagents and methods to yield information about a blood sample. Such methods are well known in the art and may be based on teachings of, e.g. Tietz Textbook of Clinical Chemistry, 3d Ed., Sec. V, pp. 776-78 (Burtis &amp; Ashwood, Eds., W. B. Sanders Company, Philadelphia, 1999); U.S. Pat. No. 5,997,817 to Chrismore et al. (Dec. 7, 1999); U.S. Pat. No. 5,059,394 to Phillips et al. (Oct. 22, 1991); U.S. Pat. No. 5,001,054 to Wagner et al. (Mar. 19, 1991); and U.S. Pat. No. 4,392,933 to Nakamura et al. (Jul. 12, 1983), the teachings of which are hereby incorporated by reference, as well as others.

Analyte detecting member may include tests in the sample test chamber that test electrochemical properties of the blood, or they may include optical means for sensing optical properties of the blood (e.g. oxygen saturation level), or they may include biochemical reagents (e.g. antibodies) to sense properties (e.g. presence of antigens) of the blood. The analyte detecting member may comprise biosensing or reagent material that will react with an analyte in blood (e.g. glucose) or other body fluid so that an appropriate signal correlating with the presence of the analyte is generated and can be read by the reader apparatus. By way of example and not limitation, analyte detecting member may “associated with”, “mounted within”, or “coupled to” a chamber or other structure when the analyte detecting member participates in the function of providing an appropriate signal about the blood sample to the reader device. Analyte detecting member may also include nanowire analyte detecting members as described herein.

Analyte detecting member may use potentiometric, coulometric, or other method useful for detection of analyte levels.

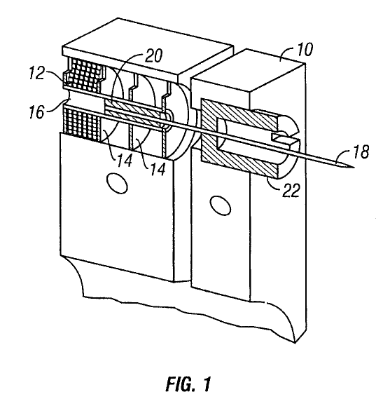

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

Referring to the embodiment of FIG. 1, the stationary iron housing 10 may contain the driver coil pack with a first coil 12 flanked by iron spacers 14 which concentrate the magnetic flux at the inner diameter creating magnetic poles. The inner insulating housing 16 isolates the penetrating member 18 and iron core 20 from the coils and provides a smooth, low friction guide surface. The penetrating member guide 22 further centers the penetrating member 18 and iron core 20. The penetrating member 18 is protracted and retracted by alternating the current between the first coil 12, the middle coil, and the third coil to attract the iron core 20. Reversing the coil sequence and attracting the core and penetrating member back into the housing retracts the penetrating member. The penetrating member guide 22 also serves as a stop for the iron core 20 mounted to the penetrating member 18.

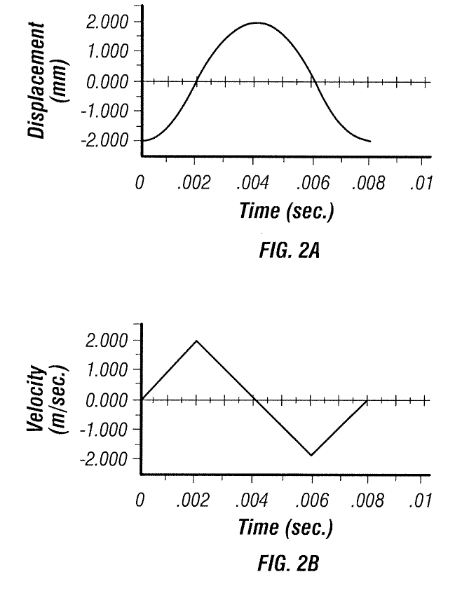

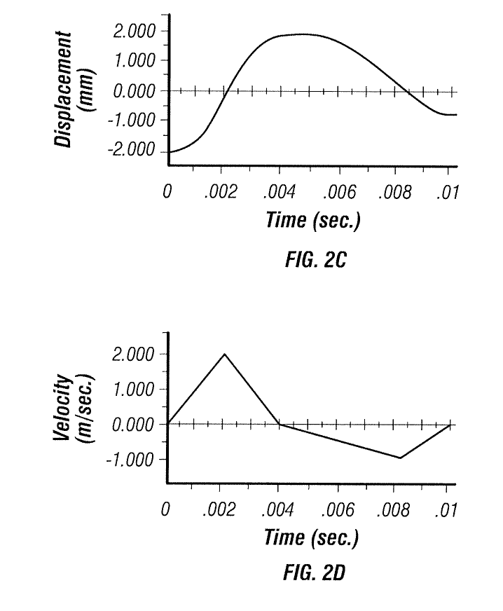

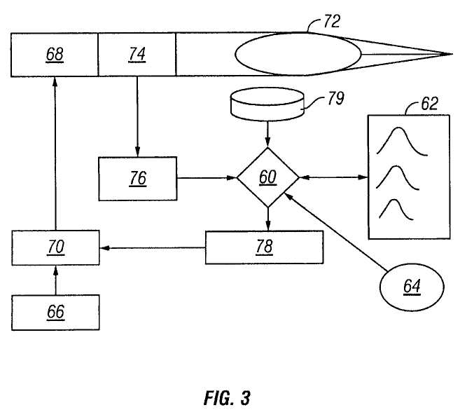

As discussed above, tissue penetration devices which employ spring or cam driving methods have a symmetrical or nearly symmetrical actuation displacement and velocity profiles on the advancement and retraction of the penetrating member as shown in FIGS. 2 and 3. In most of the available lancet devices, once the launch is initiated, the stored energy determines the velocity profile until the energy is dissipated.

Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 2C which illustrates an embodiment of a controlled displacement profile and FIG. 2D which illustrates an embodiment of a the controlled velocity profile. These are compared to FIGS. 2A and 2B, which illustrate embodiments of displacement and velocity profiles, respectively, of a harmonic spring/mass powered driver. Reduced pain can be achieved by using impact velocities of greater than about 2 m/s entry of a tissue penetrating element, such as a lancet, into tissue. Other suitable embodiments of the penetrating member driver are described in commonly assigned, copending U.S. patent application Ser. No. 10/127,395 filed Apr. 19, 2002 and previously incorporated herein.

FIG. 3 illustrates the operation of a feedback loop using a processor 60. The processor 60 stores profiles 62 in non-volatile memory. A user inputs information 64 about the desired circumstances or parameters for a lancing event. The processor 60 selects a driver profile 62 from a set of alternative driver profiles that have been preprogrammed in the processor 60 based on typical or desired tissue penetration device performance determined through testing at the factory or as programmed in by the operator. The processor 60 may customize by either scaling or modifying the profile based on additional user input information 64. Once the processor has chosen and customized the profile, the processor 60 is ready to modulate the power from the power supply 66 to the penetrating member driver 68 through an amplifier 70. The processor 60 may measure the location of the penetrating member 72 using a position sensing mechanism 74 through an analog to digital converter 76 linear encoder or other such transducer. Examples of position sensing mechanisms have been described in the embodiments above and may be found in the specification for commonly assigned, copending U.S. patent application Ser. No. 10/127,395, filed Apr. 19, 2002 and previously incorporated herein. The processor 60 calculates the movement of the penetrating member by comparing the actual profile of the penetrating member to the predetermined profile. The processor 60 modulates the power to the penetrating member driver 68 through a signal generator 78, which may control the amplifier 70 so that the actual velocity profile of the penetrating member does not exceed the predetermined profile by more than a preset error limit. The error limit is the accuracy in the control of the penetrating member.

After the lancing event, the processor 60 can allow the user to rank the results of the lancing event. The processor 60 stores these results and constructs a database 80 for the individual user. Using the database 79, the processor 60 calculates the profile traits such as degree of painlessness, success rate, and blood volume for various profiles 62 depending on user input information 64 to optimize the profile to the individual user for subsequent lancing cycles. These profile traits depend on the characteristic phases of penetrating member advancement and retraction. The processor 60 uses these calculations to optimize profiles 62 for each user. In addition to user input information 64, an internal clock allows storage in the database 79 of information such as the time of day to generate a time stamp for the lancing event and the time between lancing events to anticipate the user's diurnal needs. The database stores information and statistics for each user and each profile that particular user uses.

In addition to varying the profiles, the processor 60 can be used to calculate the appropriate penetrating member diameter and geometry suitable to realize the blood volume required by the user. For example, if the user requires about 1-5 microliter volume of blood, the processor 60 may select a 200 micron diameter penetrating member to achieve these results. For each class of lancet, both diameter and lancet tip geometry, is stored in the processor 60 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

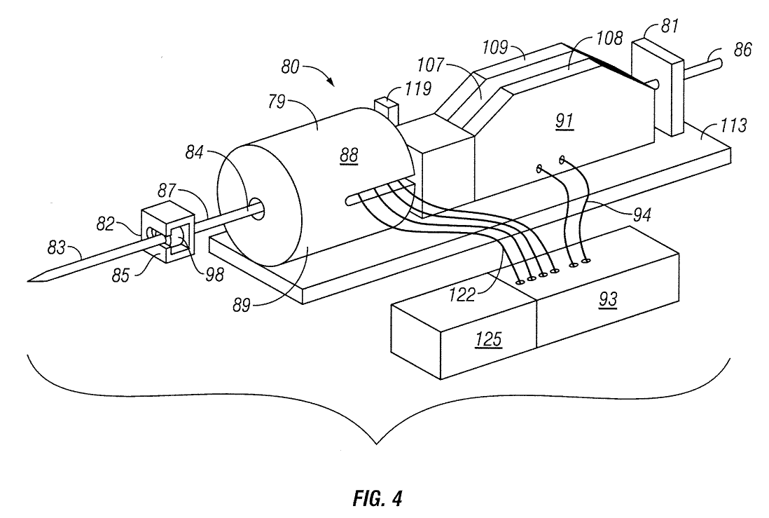

FIG. 4 illustrates an embodiment of a tissue penetration device, more specifically, a lancing device 80 that includes a controllable driver 179 coupled to a tissue penetration element. The lancing device 80 has a proximal end 81 and a distal end 82. At the distal end 82 is the tissue penetration element in the form of a penetrating member 83, which is coupled to an elongate coupler shaft 84 by a drive coupler 85. The elongate coupler shaft 84 has a proximal end 86 and a distal end 87. A driver coil pack 88 is disposed about the elongate coupler shaft 84 proximal of the penetrating member 83. A position sensor 91 is disposed about a proximal portion 92 of the elongate coupler shaft 84 and an electrical conductor 94 electrically couples a processor 93 to the position sensor 91. The elongate coupler shaft 84 driven by the driver coil pack 88 controlled by the position sensor 91 and processor 93 form the controllable driver, specifically, a controllable electromagnetic driver.

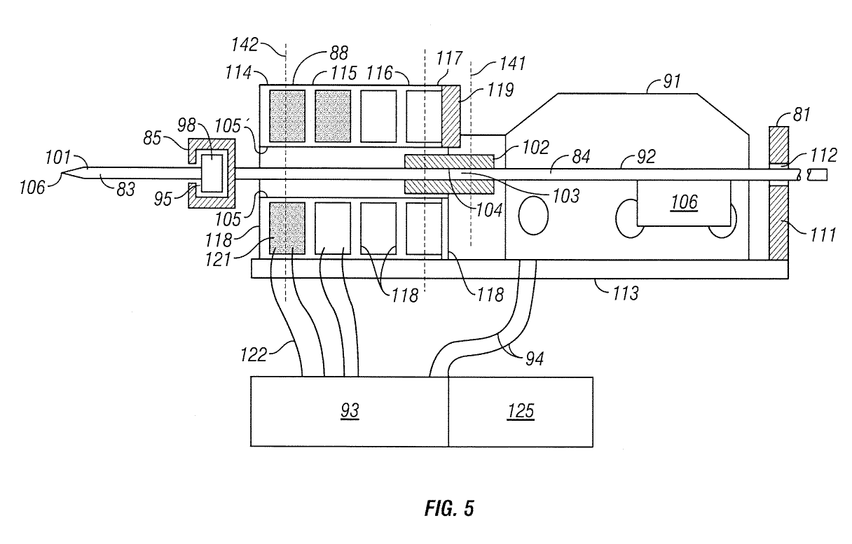

Referring to FIG. 5, the lancing device 80 can be seen in more detail, in partial longitudinal section. The penetrating member 83 has a proximal end 95 and a distal end 96 with a sharpened point at the distal end 96 of the penetrating member 83 and a drive head 98 disposed at the proximal end 95 of the penetrating member 83. A penetrating member shaft 201 is disposed between the drive head 98 and the sharpened point 97. The penetrating member shaft 201 may be comprised of stainless steel, or any other suitable material or alloy and have a transverse dimension of about 0.1 to about 0.4 mm. The penetrating member shaft may have a length of about 3 mm to about 50 mm, specifically, about 15 mm to about 20 mm. The drive head 98 of the penetrating member 83 is an enlarged portion having a transverse dimension greater than a transverse dimension of the penetrating member shaft 201 distal of the drive head 98. This configuration allows the drive head 98 to be mechanically captured by the drive coupler 85. The drive head 98 may have a transverse dimension of about 0.5 to about 2 mm.

A magnetic member 102 is secured to the elongate coupler shaft 84 proximal of the drive coupler 85 on a distal portion 203 of the elongate coupler shaft 84. The magnetic member 102 is a substantially cylindrical piece of magnetic material having an axial lumen 204 extending the length of the magnetic member 102. The magnetic member 102 has an outer transverse dimension that allows the magnetic member 102 to slide easily within an axial lumen 105 of a low friction, possibly lubricious, polymer guide tube 105′ disposed within the driver coil pack 88. The magnetic member 102 may have an outer transverse dimension of about 1.0 to about 5.0 mm, specifically, about 2.3 to about 2.5 mm. The magnetic member 102 may have a length of about 3.0 to about 5.0 mm, specifically, about 4.7 to about 4.9 mm. The magnetic member 102 can be made from a variety of magnetic materials including ferrous metals such as ferrous steel, iron, ferrite, or the like. The magnetic member 102 may be secured to the distal portion 203 of the elongate coupler shaft S4 by a variety of methods including adhesive or epoxy bonding, welding, crimping or any other suitable method.

Proximal of the magnetic member 102, an optical encoder flag 206 is secured to the elongate coupler shaft 84. The optical encoder flag 206 is configured to move within a slot 107 in the position sensor 91. The slot 107 of the position sensor 91 is formed between a first body portion 108 and a second body portion 109 of the position sensor 91.

The slot 107 may have separation width of about 1.5 to about 2.0 mm. The optical encoder flag 206 can have a length of about 14 to about 18 mm, a width of about 3 to about 5 mm and a thickness of about 0.04 to about 0.06 mm.

The optical encoder flag 206 interacts with various optical beams generated by LEDs disposed on or in the position sensor body portions 108 and 109 in a predetermined manner. The interaction of the optical beams generated by the LEDs of the position sensor 91 generates a signal that indicates the longitudinal position of the optical flag 206 relative to the position sensor 91 with a substantially high degree of resolution. The resolution of the position sensor 91 may be about 200 to about 400 cycles per inch, specifically, about 350 to about 370 cycles per inch. The position sensor 91 may have a speed response time (position/time resolution) of 0 to about 120,000 Hz, where one dark and light stripe of the flag constitutes one Hertz, or cycle per second. The position of the optical encoder flag 206 relative to the magnetic member 102, driver coil pack 88 and position sensor 91 is such that the optical encoder 91 can provide precise positional information about the penetrating member 83 over the entire length of the penetrating member's power stroke.

An optical encoder that is suitable for the position sensor 91 is a linear optical incremental encoder, model HEDS 9200, manufactured by Agilent Technologies. The model HEDS 9200 may have a length of about 20 to about 30 mm, a width of about 8 to about 12 mm, and a height of about 9 to about 11 mm. Although the position sensor 91 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like.

A coupler shaft guide 111 is disposed towards the proximal end 81 of the lancing device 80. The guide 111 has a guide lumen 112 disposed in the guide 111 to slidingly accept the proximal portion 92 of the elongate coupler shaft 84. The guide 111 keeps the elongate coupler shaft 84 centered horizontally and vertically in the slot 102 of the optical encoder 91.

The driver coil pack 88, position sensor 91 and coupler shaft guide 111 are all secured to a base 113. The base 113 is longitudinally coextensive with the driver coil pack S8, position sensor 91 and coupler shaft guide 111. The base 113 can take the form of a rectangular piece of metal or polymer, or may be a more elaborate housing with recesses, which are configured to accept the various components of the lancing device 80.

As discussed above, the magnetic member 102 is configured to slide within an axial lumen 105 of the driver coil pack 88. The driver coil pack 88 includes a most distal first coil 114, a second coil 115, which is axially disposed between the first coil 114 and a third coil 116, and a proximal-most fourth coil 117. Each of the first coil 114, second coil 115, third coil 116 and fourth coil 117 has an axial lumen. The axial lumens of the first through fourth coils are configured to be coaxial with the axial lumens of the other coils and together form the axial lumen 105 of the driver coil pack 88 as a whole. Axially adjacent each of the coils 114-117 is a magnetic disk or washer 118 that augments completion of the magnetic circuit of the coils 114-117 during a lancing cycle of the device 80. The magnetic washers 118 of the embodiment of FIG. 5 are made of ferrous steel but could be made of any other suitable magnetic material, such as iron or ferrite.

The outer shell 89 of the driver coil pack 88 is also made of iron or steel to complete the magnetic path around the coils and between the washers 118. The magnetic washers 118 have an outer diameter commensurate with an outer diameter of the driver coil pack 88 of about 4.0 to about 8.0 mm. The magnetic washers 118 have an axial thickness of about 0.05, to about 0.4 mm, specifically, about 0.15 to about 0.25 mm.

Wrapping or winding an elongate electrical conductor 121 about an axial lumen until a sufficient number of windings have been achieved forms the coils 114-117. The elongate electrical conductor 121 is generally an insulated solid copper wire with a small outer transverse dimension of about 0.06 mm to about 0.88 mm, specifically, about 0.3 mm to about 0.5 mm. In one embodiment, 32 gauge copper wire is used for the coils 114-117. The number of windings for each of the coils 114-117 of the driver pack 88 may vary with the size of the coil, but for some embodiments each coil 114-117 may have about 30 to about 80 turns, specifically, about 50 to about 60 turns. Each coil 114-117 can have an axial length of about 1.0 to about 3.0 mm, specifically, about 1.8 to about 2.0 mm. Each coil 114-117 can have an outer transverse dimension or diameter of about 4.0, to about 2.0 mm, specifically, about 9.0 to about 12.0 mm. The axial lumen 105 can have a transverse dimension of about 1.0 to about 3.0 mm.

It may be advantageous in some driver coil 88 embodiments to replace one or more of the coils with permanent magnets, which produce a magnetic field similar to that of the coils when the coils are activated. In particular, it may be desirable in some embodiments to replace the second coil 115, the third coil 116 or both with permanent magnets. In addition, it may be advantageous to position a permanent magnet at or near the proximal end of the coil driver pack in order to provide fixed magnet zeroing function for the magnetic member (Adams magnetic Products 23A0002 flexible magnet material (800) 747-7543).

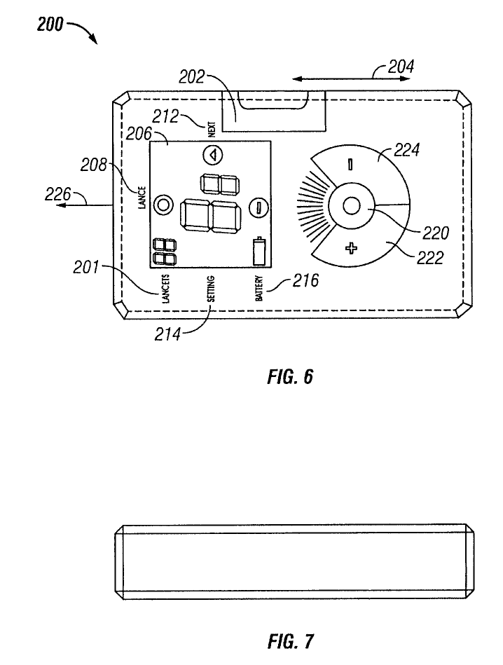

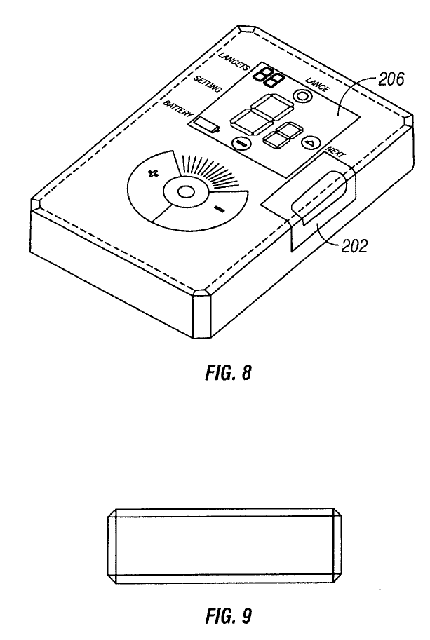

Referring now to the embodiment shown in FIGS. 6 through 9, various view of a housing 200 according to the present invention will now be described. FIG. 6 is a top view of the housing 200. The housing 200 includes a slide 202 which is movable as indicated by arrow 204. An visual display 206 may be included on the housing 200. The display 206 may have indicators that correspond to markings 208,210, 212,214, and 216 on the housing 200. These indicators may be, but are not limited to, icons, numbers, words, colors, shapes, or other visual cue that may be displayed, flashed, faded, moved, or animated to communicate information to the user. A button 220 may also be included on the housing. A second button 222 and a third button 224. As seen in FIG. 6, the buttons 222 and 224 may have markings to provide an indication of their use. In one embodiment, the buttons 222 and 224 may be used to adjust lancing performance, such as but not limited to, lancing depth, lancing speed, dwell time, or any other lancing parameter as discussed herein. Button 220 may be used for actuating the penetrating member in the direction indicated by arrow 226 to create a wound in tissue.

FIGS. 7,8, and 9 show other views of the housing 200.

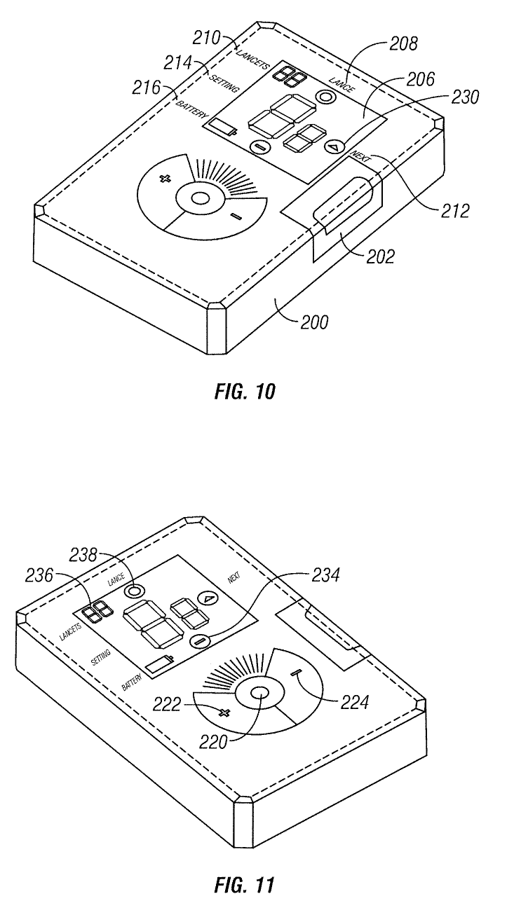

Referring now to FIGS. 10 and 11, still further views of an embodiment of the present invention is shown. In FIG. 10, the housing 200 is shown with markings 208, 210,212, 214, and 216. Each marking may correspond with some type of visual indicator on display 206. This combination of markings (which may be permanent) on the housing 200 gives a user a greater sense of security or comfort in using the lancing device. The indicators on the display 206 may be placed next to, adjacent, or in proximity to the various markings associated with the indicators. As a nonlimiting example, the circled arrow indicator 230 will flash when the user needs to move the slider 202 as indicated by arrow 232. In some embodiments, the arrow 230 will move in the direction that the slider should be moved.

As seen in FIG. 11, when an lancing performance setting should be changed, a plus or minus sign 234 will be shown. A user may then adjust the performance setting by pressing buttons 222 or 224 as appropriate. The number of penetrating members or lancets may be displayed by indicator 236. When it is time to actuate the lancet for fluid sampling, an indicator 238 may appear. The indicator may be in the shape of the button 220. In other embodiments, it may be text, the shape of a lancet, an arrow, or other indicator associated with lancing.

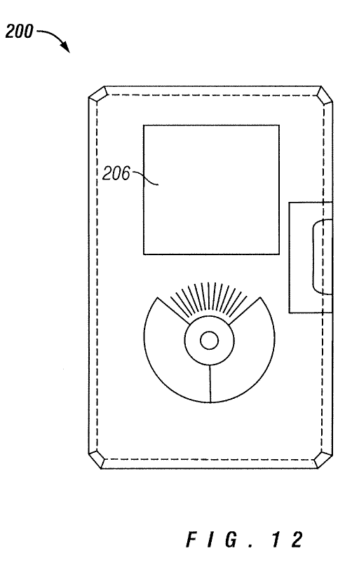

Referring now to FIG. 12, an embodiment of the present invention is shown without markings on the housing 200. In this embodiment, the visual indicators will appear on the display 206. In a nonlimiting example, the display 206 may be an LCD display, a backlit display, an LCOS display, or a device for displaying icons and/or numerals.

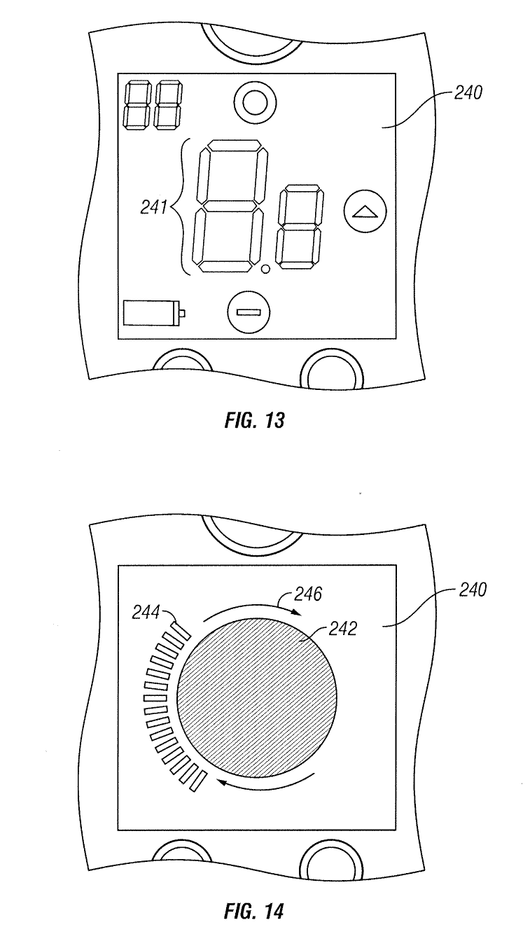

Referring now to FIG. 13, a still further embodiment of the present invention will be described. A display 240 is shown and this display may be positioned anywhere on a housing, such as but not limited to a housing 200. The display 240 may be positioned closer to the center of the housing 200. By way of example and not limitation, the display may placed on a housing having a circular, square, cylindrical, hexagonal, triangular, oval, ergonomically curved-to-fit-the-hand shape, or polygonal shape. The housing may be made of more than one material, such as a rubber bottom surface, rubber bottom half, or rubber edges to facilitate handling an grip by the user. The housing may be textured, have ribs, or other contour to ease handling by the user.

As seen in FIG. 13, lancing setting 241 may be appear on the display 240 to assist the user in determining how the lancing device is set to operate. In one embodiment, the numeral displayed may be a numerical representation of lancing depth.

The number may be on a scale of some sort, such as in one embodiment, between the depths of 0.0 to 9.9. In another nonlimiting example, the setting 241 may represent the speed setting of the lancet or penetrating member. The speed setting may be selected, in one embodiment, for an inbound path and an outbound path. Of course, other lancing parameters as discussed herein or in the referenced applications may be represented by the settings 241.

Referring now to FIG. 14, embodiments of the present invention may have a display 240 that includes a screen saver mode. In the present nonlimiting example, the screen saver is a circle 242 that may move about on the screen. In other embodiments, the screen saver includes a plurality of bars 244 that moves in a circular pattern as indicated by arrow 246. The bars 244 may of course move in other paths besides circular, such as but not limited to figure-eight, triangular, square, etc. Other screepsaver patterns as known in the art such as “spacewalp” or others as seen in many personal computer monitor displays may also be adapted for use with the present display 240.

The display can show a pattern that may optionally repeat over a period of time and wherein the pattern indicates to the user that the device is in a standby mode awaiting user interaction. In one nonlimiting example, this screensaver may come on after 30 seconds, 45 seconds, or 60 seconds of non-use by the user. As soon as a user touches a button or other input device, the screensaver will disappear. The display 240 may also be a touch sensitive display as known in the art. Some embodiments may have the screen saver shut off the display after a period of no interaction with the user. For example and not limitation, the display may be turned off 1, 2, 3, 4, 5, 6, 7, 8, 9, 10, 11, 12, 13, 14, 15, or more minutes after the screen saver is activated and there is no user input or interaction with the device.

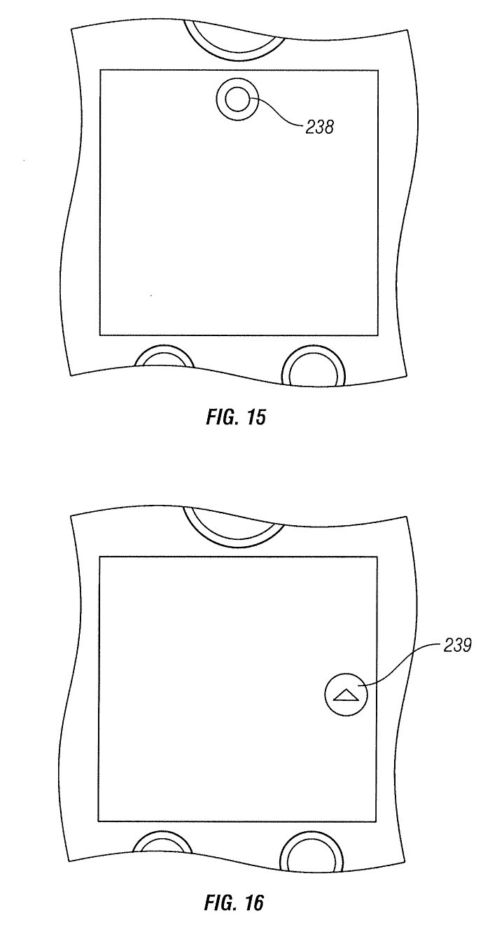

FIGS. 15,16, and 17 show that icons may be displayed individually on the display 240 to focus the users attention on the task at hand. As a nonlimiting example, the indicator 238 in FIG. 15 may appear, flash, or animate to show the it is time for the lancet to be actuated. FIG. 16 shows an icon 239 that it is time to move the slider.

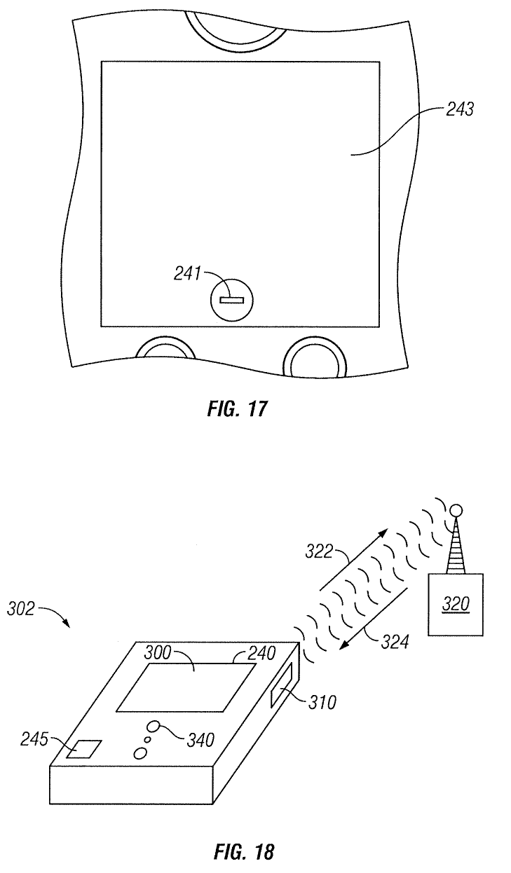

FIG. 17 shows an icon 241 that it is time to adjust the lancet setting. Any of the icons shown in the above figures may be used singly or in any combination, multiple combination, or in some sequence to provide information to the user. Some embodiments may flash an icon such as a disc or word which tell the user that the number of penetrating members has run out and it is time to change the disposable cartridge. Other embodiments may have screen 240 display a yellow background 243 when it has less than 10 penetrating members left and then a red background when it has less than 5 penetrating members left. Other may display the count in red numerals or symbols. The numbers may vary. Some may display the change in color with the yellow background when only five penetrating members are left. Others may have the red background when only 1, 2 or 3 penetrating members are left. By way of example and not limitation, in some embodiments, the background is normally green. Other embodiments may have a neutral color on display 240 when enough penetrating members are present. Still other device may have separate color area 245 that is green, yellow, and/or red depending on the number of penetrating members remaining.

Referring now to FIG. 18, a still further embodiment of the present invention will now be described. This embodiment of the present invention describes a user interface 300 that may be uploaded to the device 302. In this particular embodiment, the user interface 300 will be shown on a display 240. The device 302 may be an integrated sampling device such as, but not limited to, that shown in commonly assigned, copending U.S. patent application Ser. No. 10/421,196, filed fully incorporated herein by reference for all purposes. In the present embodiment, a wireless communication chip or processor 310 may be included in the device 302. The device 302 can then communicate with a base station 320 that may include a server or be coupled to a server. As seen in FIG. 18, information can be uploaded as indicated by arrow 322 and information can be downloaded as indicated by arrow 324. Other embodiments may use a data port 325 (shown in phantom) such as but not limited to a USB port, Firewire port, infrared port, Bluetooth connection, or other data connection. The ports may lead to a processor in the device.

For example and not limitation, a user may upload information about the user to the basestation 320 which then sends down the appropriate user interface based on the type of user who will be using the device 302. In one embodiment, the downloaded information may be a program such as but not limited to a java applet. The applet may be have a user interface for that very person, that class of users, for disease status, or the like.

As a nonlimiting example, the user interface may be for child who has juvenile diabetes.

The user interface may be varied based on the users education, familiarity with disease, or the like.

In yet another embodiment of the present invention, each user may be classified using questions that the meter displays. These questions may be graphical in nature (displaying pictures, drawings, or the like) or they may be textual questions. The questions may be used to determine a user personality type, such as but not limited to Myers Briggs, and the questions may be used to determine user preferences. The personality information may be used to determine color, shape, or other feature that the user will prefer for the analyte measurement device. These questions may be shown on display 240 for the user to answer. By way of example and not limitation, the display may be monochrome or a color display. Some embodiments may include a speaker and/or a microphone for presenting and receiving audio information to/from the user.

In this embodiment where the user interface 300 is modifiable, the user is not hindered by a user interface 300 that is too simple or too advanced for the user. The device 302 has a substantially universal hardware. As mentioned, the device 302 may include a chip 310 suitable for wireless communications such as those used for cellular phone communication. This makes the device 302 a wireless meter, among other things.

The device 302, in one embodiment, does not include the capability to call or receive telephone calls. The device 302 uses the chip 310 to send and receive information, but not calls. It should be understood, however, that in some embodiments, calls can be received.

In one particular embodiment, to initialize the device 302, the user answers a series of questions. These questions may include but are not limited to the patient's name, patient's age, patient's years with diabetes, etc. . . . to determine or classify the user.

There may also be a questionnaire to determine user personality. The device can also include other follow-up questions etc. . . . based on the users answers to previous questions in the questionnaire. The user may use buttons 340 on the device 302 to respond to questions.

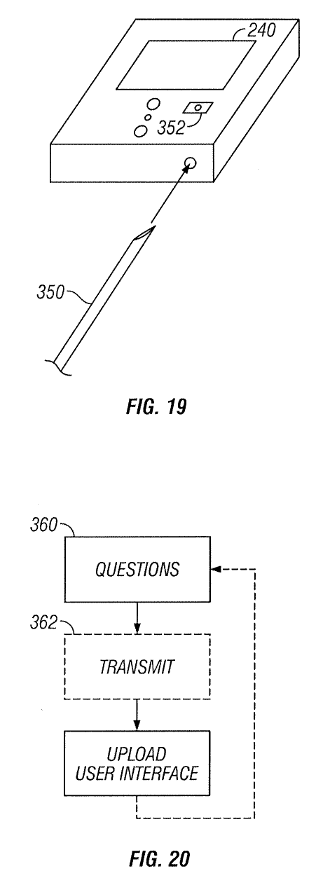

In other embodiments, as seen in FIG. 19, a stylet 350 may be used in a fashion similar that of PDA devices to enter information on a display 240. The device 302 may optionally be configured for use with a voice-recognition device 352 (with or without the stylet).

As discussed, the interface 300 can be uploaded. Having communications allows the interface to be upgraded or customized. In one embodiment, the present invention is 1) independent of hardware platform and 2) flexible in terms of the user interface (can be individualized).

The various user interfaces 300 can be widely varied. FIGS. 13-17 are some nonlimiting examples. There can be easily hundreds of designs. The designs could vary in graphic and in text. By way of example and not limitation, color, shape form and function for each of the 16 Myers Briggs groupings may segmented. Inside each of those 16 categories of personality type, there may be different subgroupings and then 5 education level in each subgroup (experienced or advanced). For example and not limitation, every second year (or some other time period) perhaps the user learns more or wants more information. This is workable with a changeable or uploadable interface.

The time period may be predetermined by a factory setting. Some may be set by the user.

By way of example and not limitation, this may be done by a java applet that is sent down to the handheld device 302. The interface may also connect via cellphone or wireless technology. This downloads applets or other software applications to the meter device. It should be understood that in some embodiments, instead of wireless, a wired connection may be used. Some devices 302 may include a plurality of interfaces loaded into the device that the user can choose from without having to do a download. By way of example and not limitation, each user interface may have a different design theme.

The device 302 could download or provide several user interfaces for the user to choose from.

Referring now to FIG. 20, a flow chart of one embodiment of the process is shown. The initial version, the user should perform a questionnaire as indicated by reference numeral 360. The questionnaire may be a single questionnaire or multiple questionnaires. They may target specific information (personality, user age and history, etc. . . . ). After the questionnaire, the information may be transmitted as indicated by number 362. Some embodiments, however, may not include this transmission step. The device 302 may be able to process the information and then provide some user interfaces already provided with the device. In other embodiments, transmission may be delayed until signal quality to the base station 320 is better. Step 364 shows the step of loading up the user interface. As discussed, the user interface may be a program or applet selected by a server that the base station 320 then transmits to the device 302.

In other embodiments, the user may ask for certain upgrades over time. The upgrades may include more interactiveness or more reminders. As a nonlimiting example, reminders could be sent such as, “Hello User, you did not measure glucose today, is there something wrong with your meter?” These reminders may be shown on the display 240. These device usage-based alerts may help to keep a user on a testing regime. The alert may be presented by audio information. Some embodiments may include a vibratory device to get the users attention. Others may use LEDs on the device or through a clear or translucent portion of the housing to obtain the users attention.

Some devices may have all the housing as clear or translucent. Others may have the top half of the housing as clear or translucent. The device could send reminders, interactively. The interface can be customized based on the patient's conditions. If the user does not have cellular coverage, there are backups (cradle or other method). The device could also be adapted in some embodiments for use with WI-FI standards used for broadband internet communications.

As mentioned, the user interface may be customized for each user or class of users. The user interface could be varied as follows. The magnitude of change may include, but are not limited to having more statistics or appointments or how often a day to measure or track whether they tested today (track testing history) they may see time to test. This could be any type of interaction or information to help the user.

As a nonlimiting example, the interfaces could designed for 10 subgroups within each personality category or vice versa. By way of example and not limitation, some examples of subgroups include gestational, type II, type I, type I unstable/brittle (glucose goes wild), juvenile, type II, type I for children, pump users, newly diagnosed (adults and children), and there are the high risk/at risk group where testing is recommended. The users may be advanced or not advanced at all.

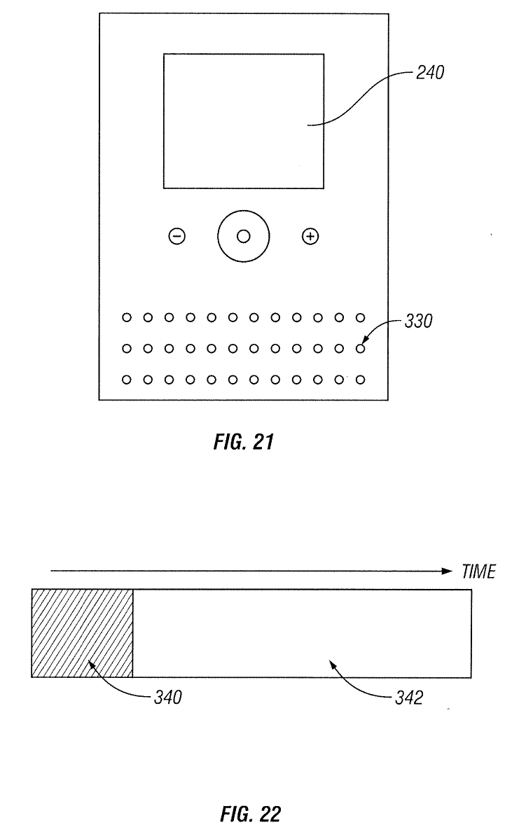

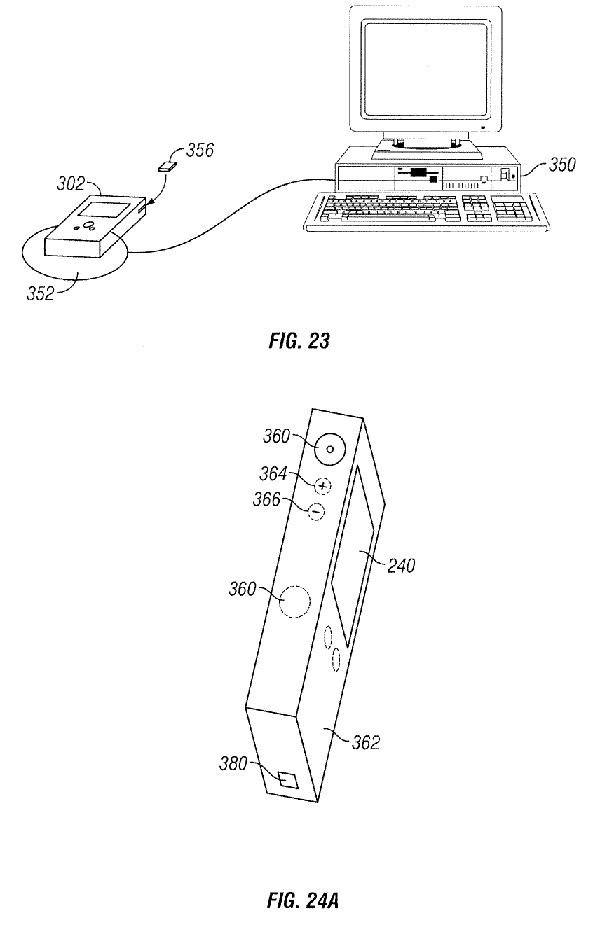

Referring now to FIG. 21, a device with a keyboard 330 may be included. This may facilitate the answering of the questionnaire. By way of example and not limitation, this embodiment may also include three buttons (one for lancing and two for positive or negative adjustments). The questionnaire may be formulated to find out information such as but not limited to the following: to find out what age, what type of diabetes, what education they have, their history in the family. The questionnaire may also used to classify their disease understanding, the age level, the relationship level, history level, support, healthcare provider, location they are in, what type of support, or what type of interface is preferred. The questionnaire may include any one of the following questions: What does the user prefer in terms of features? What is the users eye vision? Does the user want big numbers or fonts? Does the user want marketing or new product reminders? Does the user want reminders when the device runs out? Does the user want notices of diabetes group meetings in the area? Does the user want to get invited to conventions? Some elements could be once a month questions. For example and not limitation, there could be new information once a month and the device may ask the user if he or she wants more information. It should be understood the timing of information could be but are not limited to daily, weekly, monthly, or yearly settings.

Referring now to FIG. 22, today it takes 1.5 minutes from preparing to lancing to getting the glucose result. The measurement time is only 5-20 seconds, but all the other steps take up the 1.5 minutes. The new process may only take 10 seconds as indicated by area 340. The additional time 342 can also be filled with information that has not been given so far. So, after a glucose meter reading is displayed, other information could be displayed afterwards. For example and not limitation, the information may be statistics on user testing, on how they are doing with glucose or healthwise, their glucose readings for the last 5 measurements, readings for some desired number of measurements, a graph of their glucose levels over a desired length of time, glucose level based on time of day, showing how much over or under their glucose level has been for the last 5, 10, other number of measurements from a desired glucose level, or other information on disease management. The perception is that testing will still take a long time. More valuable information can be delivered with the result since the user is accustomed to waiting. This embodiments delays display of the glucose reading by a predetermined amount of time. Other embodiments may display their glucose level reading from the measurement as soon as it is available and show the above other information after the result has displayed for 5-10 or other number of seconds. Some embodiments may show the current glucose or analyte reading while also showing the glucose management information. The glucose reading could be delayed while other information is displayed first. In some embodiments, the reading may be delayed from being shown on display 240 for a whole minute, 30 seconds, or other set time. There may be no delay or additional information. The user may adhere to testing better since it takes less time. The use model is not interfered with since the users are use to the amount of time or amount of time lag for a glucose measurement to be displayed.

In a yet further embodiment, the user may answer the questionnaire at the time of purchase or shortly there after. The questionnaire may be on paper or a ScanTron type form that is processed by a pharmacist or mailed to a central location at time of sale, just before sale, or after sale. As seen in FIG. 22, some embodiments may have a computer 350 present to allow a user to answer the questions. Based on the responses, the correct user interface will be downloaded to the device 302 sitting in an optional docking station 352. In other embodiments, a pharmacist may upload the correct UI by connecting the device to his computer. In other embodiments, the device may initialize (prior to being customized based on questionnaire response) with a standard interface which is then customized when the questionnaire information is processed. In one embodiment, the standard user interface would allow the user to obtain analyte readings. In yet another embodiment, the questionnaire may tell the user to select from a variety of chips or cartridges 356 provided with the device 302. In yet other embodiments, a code may be provided for the user to enter which will then select the correct UI already loaded into the processor. By way of example and not limitation, the code may be selected from instructions with the device, displayed on the computer 350 based on answers to the questionnaire, or given orally over the phone or by a retailer. It may also be transmitted wirelessly by the methods described herein.

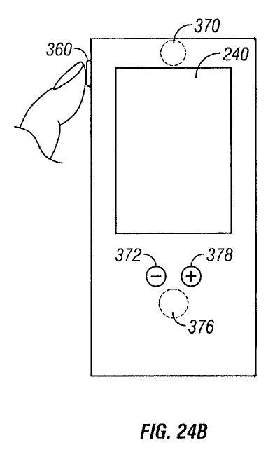

Referring now to FIG. 24A, a side perspective view is shown of yet another embodiment of the present invention. This embodiment shows that the button 360 to activate lancing may be located on a side of the housing 362. As shown in phantom, adjustment buttons 364 and 366 may also be included on the side in some embodiments.

Some embodiments may have the button 360 in a concave portion so that the button does not protrude or it may protrude (as shown in FIG. 24). Some embodiments may move the location of button 360 on the side surface of the housing. Other embodiments may have left hand and right hand user models where the fire button is on one side or the other. Others may have fire buttons 360 on both sides of the housing.

Referring now to FIG. 24B, yet another embodiment of the present invention is <BR> <BR> shown. In this embodiment, “fire” button 360 may be located on a side of the housing similar to that of FIG. 24A. In this embodiment, another “fire” button 370 may also be included. Adjustment buttons 372 and 374 are on the surface of the housing with screen 240. The button 376 (shown in phantom) may be included in this location instead of button 370. The embodiments of FIGS. 24A, 24B, and any embodiment herein may include a “key lock” feature where the device will be locked once the sequence is punched. By way of example and not limitation, the device may involve pressing the “+” adjustment button and sequentially the fire button to lock and/or unlock the keys. This prevents accidental firings of the device. Other combinations are possible such as “−” and then the fire button. Other buttons may be used. Some may use one button on one surface of the housing and sequentially punching another button on another surface.

Some may have both buttons punched on the same surface. The device may have a port 380 to allow for battery charging.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

For example, with any of the above embodiments, features may be used with meter only devices or integrated devices that include metering and lancing. With any of the above embodiments, other programs besides those that change a user interface may also be downloaded. As a nonlimiting example, the device may download upgrades or improvements in analyte monitoring sensitivity. With any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). With any of the above embodiments, the penetrating members may be a bare penetrating member during launch.

With any of the above embodiments, the penetrating members may be bare penetrating members prior to launch as this may allow for significantly tighter densities of penetrating members. In some embodiments, the penetrating members may be bent, curved, textured, shaped, or otherwise treated at a proximal end or area to facilitate handling by an actuator. The penetrating member may be configured to have a notch or groove to facilitate coupling to a gripper. The notch or groove may be formed along an elongate portion of the penetrating member. With any of the above embodiments, the cavity may be on the bottom or the top of the cartridge, with the gripper on the other side.

In some embodiments, analyte detecting members may be printed on the top, bottom, or side of the cavities. The front end of the cartridge maybe in contact with a user during lancing. The same driver may be used for advancing and retraction of the penetrating member. The penetrating member may have a diameters and length suitable for obtaining the blood volumes described herein. The penetrating member driver may also be in substantially the same plane as the cartridge. The driver may use a through hole or other opening to engage a proximal end of a penetrating member to actuate the penetrating member along a path into and out of the tissue.

Any of the features described in this application or any reference disclosed herein may be adapted for use with any embodiment of the present invention. For example, the devices of the present invention may also be combined for use with injection penetrating members or needles as described in commonly assigned, copending U.S. patent application Ser. No. 10/127, filed Apr. 19, 2002.

An analyte detecting member to detect the presence of foil may also be included in the lancing apparatus. For example, if a cavity has been used before, the foil or sterility barrier will be punched. The analyte detecting member can detect if the cavity is fresh or not based on the status of the barrier. It should be understood that in optional embodiments, the sterility barrier may be designed to pierce a sterility barrier of thickness that does not dull a tip of the penetrating member. The lancing apparatus may also use improved drive mechanisms. For example, a solenoid force generator may be improved to try to increase the amount of force the solenoid can generate for a given current. A solenoid for use with the present invention may have five coils and in the present embodiment the slug is roughly the size of two coils. One change is to increase the thickness of the outer metal shell or windings surround the coils. By increasing the thickness, the flux will also be increased. The slug may be split; two smaller slugs may also be used and offset by ½ of a coil pitch. This allows more slugs to be approaching a coil where it could be accelerated. This creates more events where a slug is approaching a coil, creating a more efficient system.

In another optional alternative embodiment, a gripper in the inner end of the protective cavity may hold the penetrating member during shipment and after use, eliminating the feature of using the foil, protective end, or other part to retain the used penetrating member. Some other advantages of the disclosed embodiments and features of additional embodiments include: same mechanism for transferring the used penetrating members to a storage area; a high number of penetrating members such as 25, 50, 75, 100, 500, or more penetrating members may be put on a disk or cartridge; molded body about a lancet becomes unnecessary; manufacturing of multiple penetrating member devices is simplified through the use of cartridges; handling is possible of bare rods metal wires, without any additional structural features, to actuate them into tissue; maintaining extreme (better than 50 micron-lateral-and better than 20 micron vertical) precision in guiding; and storage system for new and used penetrating members, with individual cavities/slots is provided. The housing of the lancing device may also be sized to be ergonomically pleasing. In one embodiment, the device has a width of about 56 mm, a length of about 105 mm and a thickness of about 15 mm. Additionally, some embodiments of the present invention may be used with non-electrical force generators or drive mechanism. For example, the punch device and methods for releasing the penetrating members from sterile enclosures could be adapted for use with spring based launchers. The gripper using a frictional coupling may also be adapted for use with other drive technologies.

Still further optional features may be included with the present invention. For example, with any of the above embodiments, the location of the penetrating member drive device may be varied, relative to the penetrating members or the cartridge. With any of the above embodiments, the penetrating member tips may be uncovered during actuation (i.e. penetrating members do not pierce the penetrating member enclosure or protective foil during launch). The penetrating members may be a bare penetrating member during launch. The same driver may be used for advancing and retraction of the penetrating member. Different analyte detecting members detecting different ranges of glucose concentration, different analytes, or the like may be combined for use with each penetrating member. Non-potentiometric measurement techniques may also be used for analyte detection. For example, direct electron transfer of glucose oxidase molecules adsorbed onto carbon nanotube powder microelectrode may be used to measure glucose levels. Additional details related to the present invention may be found in co-owned U.S. Provisional Application Ser. No. 60/511,621 filed Oct. 14, 2003. All applications listed above are fully incorporated herein by reference for all purposes.

In one embodiment, a method is provided for periodically obtaining blood samples for testing. Lancing device 80, which is portable, is used to puncture a body part and obtain. An energy source, e.g., power supply 66, including but not limited to one or more rechargeable batteries 66 of the portable lancing device 80 is depleted. The portable lancing device 80 is periodically connected to a charging station and to recharge the energy source 66.

The lancing device 80, can have a port for recharging the power source 66 positioned in the housing 200. The power source 66 can be a battery, more particularly a rechargeable battery. The display 206 can provide an indication of battery status. A charge level indicator can be provided that displays the electrical charge level of the battery 66.

In response to an input at the display 206, user interface, an electrical input can be provided to the battery 66. The battery 66 and the driver 179 provide for the creation of a wound tract that remains open for a sufficient time to permit a flow of a body fluid to a sample chamber in the housing 200.

In another embodiment, the lancing device 80 includes a battery 66 and a port to recharge the battery 66. The penetrating member driver 179 periodically drives a penetrating member 83 to pierce the skin surface and create a wound tract. A port is periodically used to recharge the battery 66. A detected amount of stored energy remaining in the battery 66 can be displayed.

Patient information can be transferred from an external source to an electronic element of the skin lancing device.

The energy source 66 (rechargeable battery) of the portable skin lancing device is partially depleted and periodically connected to a charging station to recharge the rechargeable battery of the skin lancing device.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications, patents, and patent applications mentioned herein are incorporated herein by reference to disclose and describe the structures and/or methods in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Expected variations or differences in the results are contemplated in accordance with the objects and practices of the present invention. It is intended, therefore, that the invention be defined by the scope of the claims which follow and that such claims be interpreted as broadly as is reasonable.

1. An analyte measurement device comprising:
a housing;
a visual display on said housing, said visual display having at lease one visual indicator position next to a corresponding marking on the housing; and
a processor driving the visual display, wherein the processor runs software that is modifiable to provide a variable user interface on the visual display.
a rechargeable battery.

2. A method of periodically obtaining blood samples, comprising: periodically using an electrically powered portable lancing aid to puncture a body part and obtaining a blood sample from the body part;
at least partially depleting an energy source of the portable lancing aid; and periodically connecting the portable lancing aid to a charging station and thereby recharging the energy source of the portable lancing aid.

3. An analyte measurement device, comprising:
a housing;
a visual display on the housing;
a penetrating member configured to be coupled to an actuator;
a port for recharging a power source positioned in the housing.

4. The device of claim 3, wherein the power source is a battery.

5. The device of claim 3, wherein the power source is a rechargeable battery.

6. The device of claim 3, wherein in response to an input at the visual display user interface an electrical input is provided to the power source.

7. The device of claim 4, wherein the battery and the actuator provide for the creation of a wound tract that remains open for a sufficient time to permit a flow of a body fluid to a sample chamber in the housing.

8. The device of claim 3, wherein the power supply is an electric motor.

9. The device of claim 3, wherein the visual display is accessible from outside the housing.

10. The device of claim 4, wherein the visual display provides an indication of battery status.

11. The device of claim 5, wherein an electric motor comprises a direct current motor.

12. The device of claim 1, wherein the device is configured to hold a disposable having a plurality of penetrating members.

13. The device of claim 4, further comprising:
a charge level indicator that displays the electrical charge level of the battery

14. The system of claim 3, wherein the actuator comprises a magnetic system.

15. The system of claim 3, wherein the actuator comprises a piezoelectric system.

16. A skin lancing device, comprising:
a housing;
a visual display on said housing, said visual displaying having at least one visual indicator position next to a corresponding marking on the housing;
a processor driving the visual display, wherein the processor runs software that is modifiable to provide a variable user interface on the visual display;
a penetrating member coupled to an actuator; and
a port for recharging a battery positioned in the housing.

17. The device of claim 16, wherein the power source is a rechargeable battery.

18. The device of claim 16, wherein in response to an input at the visual display user interface an electrical input is provided to the battery.

20. The device of claim 16, wherein the battery and the actuator provide for the creation of a wound tract that remains open for a sufficient time to permit a flow of a body fluid to a sample chamber in the housing.

21. The device of claim 16, wherein the visual display is accessible from outside the housing.

22. The device of claim 16, wherein the visual display provides an indication of battery status.

23. The device of claim 16, wherein the device is configured to hold a disposable having a plurality of penetrating members.

24. The device of claim 16, further comprising:
a charge level indicator that displays the electrical charge level of the battery

25. The system of claim 16, wherein the actuator comprises a magnetic system.

26. The system of claim 16, wherein the actuator comprises a piezoelectric system.

27. A method, comprising:
providing a skin lancing device that includes a battery and a port to recharge the battery;
downloading software to the monitor wherein the software contains a selected user interface;
periodically using a driver to drive a penetrating member, pierce a skin surface and create a wound tract;
periodically using the port to recharge the battery.

28. The method of claim 27, further comprising
displaying a detected amount of stored energy remaining in the battery.

29. The method of claim 27, further comprising:
providing an audible signal.

30. The method of claim 27, further comprising:
transferring patient information from an external source to an electronic element of the skin lancing device.

31. A method of periodically obtaining blood samples, comprising:
periodically using an electrically powered portable skin lancing device to puncture a body part and obtaining a blood sample from the body part;
at least partially depleting an energy source of the portable skin lancing device; and periodically connecting the portable skin lancing device to a charging station and thereby recharging a battery of the skin lancing device.

32. The method of claim 31, further comprising
displaying a detected amount of stored energy remaining in the battery.

33. The method of claim 31, further comprising:
providing an audible signal.

34. The method of claim 31, further comprising:
transferring patient information from an external source to an electronic element of the skin lancing device.

35. A method of periodically obtaining blood samples, comprising:
periodically using an electrically powered skin lancing device to puncture a body part and obtaining a blood sample from the body part, the skin lancing device including a port for recharging a battery positioned in the skin lancing device; and
using the port to recharge the battery.

36. The method of claim 35, further comprising
displaying a detected amount of stored energy remaining in the battery.

37. The method of claim 35, further comprising:
providing an audible signal.

38. The method of claim 35, further comprising:
transferring patient information from an external source to an electronic element of the skin lancing device.

39. A method for sampling blood, comprising:
providing a skin lancing device that includes a plurality of penetrating members, a plurality of analyte sensors, a driver, a battery and a port for recharging the battery;
periodically using a driver to drive a penetrating member, pierce a skin surface and create a wound tract;
periodically using the port to recharge the battery.

40. The method of claim 39, further comprising
displaying a detected amount of stored energy remaining in the battery.

41. The method of claim 39, further comprising:
providing an audible signal.

42. The method of claim 39, further comprising:
transferring patient information from an external source to an electronic element of the skin lancing device.
